(19) United States
(12) Patent Application Publication
(10) Pub. No.: US 2002/0049447 A1
(43) Pub. Date: Apr. 25, 2002

(54) Expandable surgical fastener and method

(75) Inventor: Lehmann K. Li, Milford, CT (US)

Correspondence Address: OSTROLENK FABER GERB & SOFFEN, 1180 AVENUE OF THE AMERICAS, NEW YORK, NY, 100368403

(73) Assignee: Li Medical Technologies, Inc.

(21) Appl. No.: 09/938,111

(22) Filed: Aug. 23, 2001

Related U.S. Application Data
(60) Provisional application No. 60/228,948, filed on Aug. 29, 2000.

(51) Int. Cl. A61B 17/58

(52) U.S. Cl. 606/73

(57) ABSTRACT

A surgical fixation device for fixation in a biological medium, the device comprising a first member having an internal longitudinal bore having an internal diameter, a second shaft member slidably disposed in the internal bore of the first member, the second shaft member having a distal end having an external diameter greater than the internal diameter of the first member, the distal end comprising a cam surface for engaging with an engaging surface of the first member, the first member being expandable adjacent a distal end thereof, the second shaft member having an engagement portion at a proximal end for engagement by an actuation tool for application of a proximally directed axial force to the shaft member, whereby application of the proximally directed axial force to the shaft member causes the cam surface to slidably engage the engaging surface and thereby cause the first member to expand radially thereby securing the first member with the shaft member therein in the biological medium.

### CROSS REFERENCE TO RELATED APPLICATION

This application claims the benefit and priority of U.S. Provisional Application Ser. No. 60/228,948 filed Aug. 29, 2000 entitled “POP SCREW BONE FASTENER AND METHOD” the disclosure of which is incorporated by reference.

### BACKGROUND OF THE INVENTION

The present invention relates to the field of surgical fixation devices used for implantation into tissue, and in particular, into bone. The invention further relates to a method of implanting such devices. Surgical fixation devices can be used to attach sutures or prosthesis to bone tissue, for example.

One deficiency of current fixation devices, primarily bone screws, is that in osteoperotic bone, or bone with poor mineral density, the device's holding power is diminished. The threads of the conventional bone screw often cannot provide sufficient fixation strength given the soft medium of osteoperotic bone. Another deficiency of current screws is that the length of some screws has been extended to obtain “bi-cortical” bony purchase to gain additional fixation strength when dealing with osteoperotic bone. This has the disadvantage of reducing the number of application sites.

Modern trends in surgery include the restoration of bodily function and form, i.e., repair of anatomical structures through the use of minimally invasive surgical techniques. Performing surgery on patients with osteoperotic bone is particularly challenging since the poor quality of bone can compromise bone screw efficacy, thus affecting such intended goals as stabilization or fusion.

A known bone screw is described in U.S. Pat. No. 5,209,753 to Biedermann et al. The Biedermann et al. patent discloses a screw-in-screw type of device utilizing a central shaft to expand an outer cylinder having exterior screw threads. The center shaft has an enlarged end to cause expansion of the outer screw. The expansion is caused by turning the inner shaft in the outer screw. The inner shaft is rotatable via mating screw threads contained at the proximal end of the inner shaft and along an inner diameter of the proximal end of the outer screw. This screw-in-screw type of technology has deficiencies in that more of the outer screw cross section is lost to the threads necessary to cause rotation to move the inner shaft in the outer screw to expand the outer screw. Further, the screw-in-screw technology has additional frictional losses due to rotational friction of the internal screw threads. These additional losses require additional torque. Further, the screw-in-screw technology requires a time consuming rotational motion to set the fastener. Furthermore, the screw-in-screw technology leaves the surgeon unsure of whether the fastener has been sufficiently set in the bone and is additionally more complex to fabricate.

Other patents of interest include U.S. Pat. Nos. 5,501,695 5,326,205 to Anspach Jr., et al., U.S. Pat. No. 5,618,142 to Sonden et al., U.S. Pat. No. 4,790,304 to Rosenberg and U.S. Pat. No. 5,169,400 to Muhling et al.

The two Anspach, Jr., et al. patents, disclose an expandable rivet assembly having a sleeve with a shaft insertable therein. The shaft is pulled proximally to crush the sleeve and expand it outwardly. The sleeve includes a plurality of slots which form ribs between the slots which allow the sleeve to crush.

Although not in the surgical field, U.S. Pat. No. 5,618,142 to Sonden et al. discloses a self drilling blind rivet which is somewhat similar to the device of the Anspach Jr. et al. references although it is intended as a blind rivet for holding two materials together. The rivet crushes at the blind end to hold the materials riveted together.

U.S. Pat. No. 5,169,400 to Muhling et al. discloses a bone screw which has a non-circular channel in the center for allowing an insertion tool to be inserted. A plug member is provided for plugging the center hole in the screw member for closing off the center hole. The plug member is not used to provide an expansion effect. In order to allow high torque, the channel has a keyed shape in cross section along most of the length of the shaft.

U.S. Pat. No. 4,790,304 to Rosenberg has a sleeve and a central slidable member. The sleeve has slits at both ends and the shaft has an enlargement at a distal end and a collar at a proximal end so that both the proximal and distal ends can be expanded by suitable axial forces which can be applied by a screw device.

All of the above references suffer from deficiencies. In the case of the screw-in-screw devices they suffer from the disadvantages mentioned above. In the case of the crushable collar devices like the Anspach, Jr., et al. references, it is not clear that these devices provide the consistency necessary for bone fasteners, and furthermore, these devices are complex, requiring carefully designed crush collars. Further, the nonexpandable screw type devices do not provide the required fixation strength, particularly in soft bone.

### SUMMARY OF THE INVENTION

It is an object of the present invention to provide a surgical fastening device which avoids the deficiencies of the prior art.

In particular, it is an object of the present invention to provide a bone fastening device which can be set with a simple motion.

It is furthermore an object of the invention to provide a fixation device which is simple to manufacture.

It is furthermore an object of the present invention to provide a bone fastening device and method which is also suited for use in osteoperotic or soft bone.

It is furthermore an object of the invention to provide sufficient soft bone fixation through a “unicortical” expandible screw, i.e., a fastener which can be used to obtain fixation in primarily soft bone tissue.

The above and other objects of the present invention are achieved by a surgical fixation device for fixation in a biological medium comprising: a first member having an internal longitudinal bore having an internal diameter, a second shaft member slidably disposed in the internal bore of the first member, the second shaft member having a distal end having an external diameter greater than the internal diameter of the first member, said distal end comprising a cam surface for engaging with an engaging surface of said first member, said first member being expandable adjacent a distal end thereof, said second shaft member having an engagement portion at a proximal end for engagement by an actuation tool for application of a proximally directed axial force to said shaft member, whereby application of said proximally directed axial force to said shaft member causes said cam surface to slidably engage said engaging surface and thereby cause said first member to expand radially thereby securing said first member with said shaft member therein in the biological medium.

The objects of the invention are also achieved by a method for delivering an expandible fixation device into biological tissue comprising the steps of:

accessing and preparing an insertion site in the biological tissue;

introducing an expandable fixation device into the biological tissue in an undeployed state;

the expandable fixation device comprising an outer sleeve and an inner shaft disposed longitudinally in the outer sleeve and having a distal end which has a larger diameter than an inside diameter of the distal end of the outer sleeve; and

once the expandable fixation device is disposed in the biological tissue at a selected depth, applying a proximally directed axial force to the inner shaft without rotation of the inner shaft to expand the distal end of the outer sleeve radially into the biological tissue thereby securing the expandable fixation device in the tissue.

It is also an object of the present invention to provide such a fastening device and method which is employed with minimally invasive surgical techniques.

Other features and advantages of the present invention will be described in the following detailed description.

### BRIEF DESCRIPTION OF THE DRAWING(S)

The invention will now be described in greater detail in the following detailed description with reference to the drawings in which:

FIG. 1 is a cross sectional view of the expandable fixation device according to the present invention;

FIG. 2 is an exploded view of the expandable fixation device of FIG. 1 also showing, in greater detail, the drive shaft for setting the device;

FIG. 3 shows one embodiment of a drive tool and mechanical linkage for setting the expandable fixation device;

FIG. 3A is an end view of the distal end of the actuation tool;

FIG. 4 is a perspective view of the expandable fixation device;

FIG. 5 contrasts in view A a cross section of the device according to the present invention and in view B a cross section of a screw-in-screw type of device; and

FIG. 6 shows the expandable fixation device after deployment.

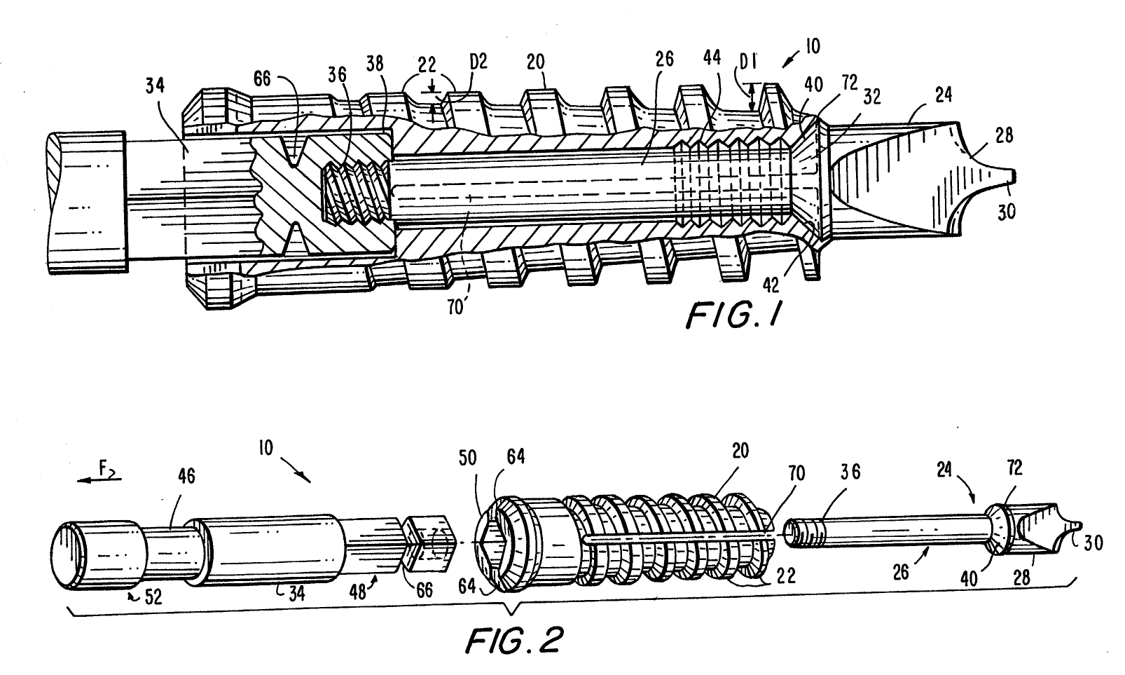

### DETAILED DESCRIPTION OF EMBODIMENTS OF THE INVENTION

With reference now to the drawings, the invention provides a surgical fastener, for example a bone fastener, shown generally by reference 10. The bone fastener comprises an outer hollow cylindrical member 20 which may have external screw threads 22 as shown. However, the device need not have screw threads. It can have some other external surface, for example a smooth surface, a serrated surface, a grooved surface, or any other type of surface adapted to achieve an expandable frictional fixation into a bone hole. Preferably, the outer diameter of the device has a taper that increases from the distal to the proximal end. As shown, if a screw thread is employed, the depth of the thread D1 is greater at the distal end than at the proximal end of the thread, as shown at D2. This assists in both the cutting threading action and the frictional engagement after expansion.

Inside the hollow member 20, a pin member 24 is slidably provided. The pin member 24 includes a shaft 26 having a distal end 28. The distal end 28 may be provided with a self tapping drill bit 30. Alternatively, the distal end of member 20 can be provided with a drill bit. The pin member 24 has an expanded portion 32 at the distal end. The expanded portion 32 has a diameter greater than the diameter of the shaft portion 26.

At its proximal end, the shaft 26 is attached to a driving shaft 34. The shaft 26 may be attached to the driving shaft 34 via threads 36, for example. However, the shaft 26 and the shaft 34 may be fixed in any suitable way, for example threads, crimping, welding, etc. In the embodiment shown, the driving shaft 34 is fastened to the shaft 26 by threads 36 and by a crimping as shown at 38. However, either alone would be adequate. The purpose of driving shaft 34 is to transfer rotational torque from the member 20 to the shaft portion 26 to rotate the drill bit 30, as will be explained below.

At the distal end, the pin member 24 has an expanded section 32 which has a conical surface 40. The hollow member 20 has a mating tapered surface 42 upon which the conical surface 40 engages. Along the inner diameter of the member 20, a plurality of grooves or serrations 44 are provided which form a one way ratchet, the purpose of which will be explained herein.

Shaft 34 is provided with a recessed portion 46 which is adapted to be grasped by an actuation device to be described later. The actuation device can be similar to the actuation tool sold by Li Medical for use with the Ultrafix surgical anchor. An example of such a grasping mechanism is shown in U.S. Pat. No. 5,843,127. Basically, the tool has a collet which is received in the recess 46 to apply a proximally directed axial force to shaft 34 to set the device. Further, shaft 34 has a portion having a keyed shape, for example a square, hexagonal or polygonal shape as shown at 48. The keyed portion of shaft 48 is adapted to be received in a like keyed opening 50 of the outer member 20. This allows the outer member 20, particularly when it is provided with screw threads 22, to drive the driving shaft 34 in rotation, which thereby drives the shaft 26 and drill bit 30 rotatably into the biological tissue.

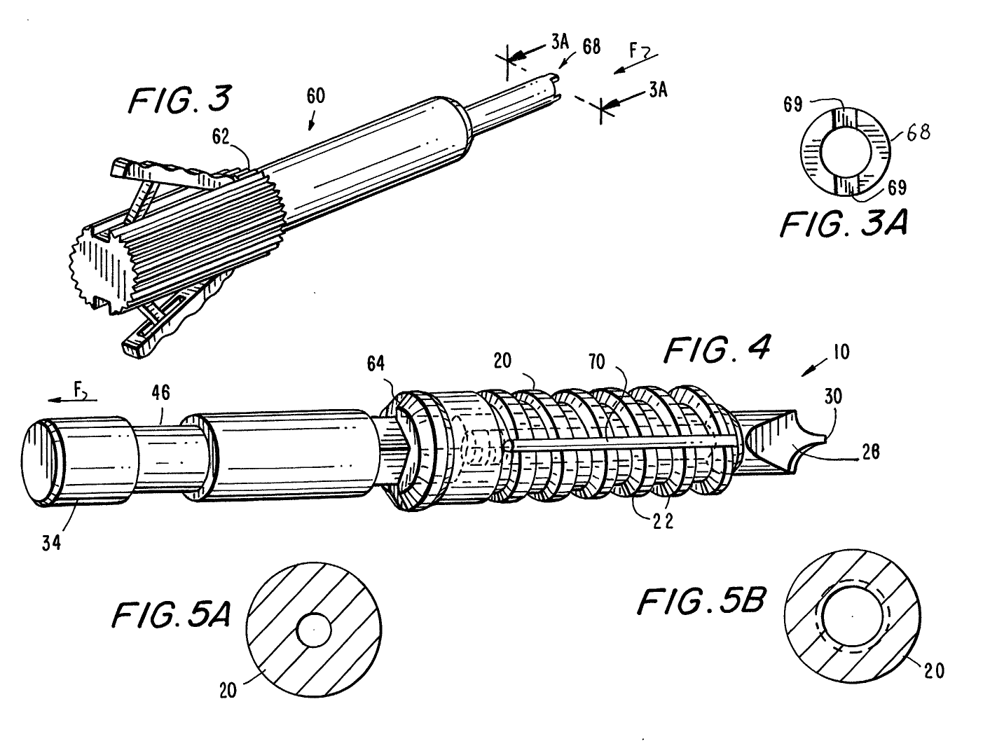

The device works as follows. An actuating tool shown in FIG. 3 and indicated by reference numeral 60 is provided over the proximal end 52 of the shaft 34 and engages the shaft at recess 46 in order to provide an axial force in the direction of the arrow F. The tool 60 has, at its distal end, a screwdriver or other keyed engagement surface. In the embodiment shown, the tool has a screwdriver end 68. The end 68 is hollow in the center to enable it to surround shaft 34. The end 68 has two opposed projections 69, similar to a spanner tool, to enable it to rotate member 20 through engagement with slots 64 of member 20. See FIG. 3A. The shaft 34 is connected via the threads 36 and/or crimp connection to the shaft 26. The threaded outer member 20 surrounds the shaft 26. The shaft 26 preferably includes a drill bit 30 at the distal end. When the actuating mechanism 60 is rotated, for example, by securing it in an electric drill (or optionally manually), the drill rotates end 68 of tool 60, thereby rotating the member 20 via engagement with slots 64. The external screw member 20, in turn, rotates the shaft 34 via the keyed portion 48 which mates with the similarly keyed opening 50 in the threaded member 20. As the drill turns the member 20, it thus turns the shaft 34 as well as the shaft 26 and the drill bit 30. The drill bit 30 therefore drills through the hard cortical layer of bone into the softer cancellous layer. At this point, power drilling is preferably stopped and further drilling in the soft cancellous layer of bone is preferably performed manually. Once the cancellous layer is reached, the screwdriver handle 62 of the actuating device 60 can be turned manually thereby causing the drill point 30 to drill into the soft cancellous layer and at the same time the external screw threads 22 of the element 20 self tap into the previously bored opening in the cortical layer and then into the soft cancellous layer.

The combination of the drill bit 30 with the threaded member 20 allows the bone fastener to be inserted in one operation. It is not necessary to pre-drill a bore hole in the bone. The drill bit 30 drills the bore hole in the bone at the same time that the fastener is being inserted.

The square or keyed portion 48 of the shaft 34 is provided to ensure that enough torque can be transferred from the member 20 to drill through the hard cortical layer. As with the preferred power drilling through the cortical layer, the manual transfer of torque is from the member 20 via keyed portions 48 and 50 to the drill bit 30 to insert the fastener fully to the desired depth through the cortical layer into the cancellous layer. Once the fastener has been inserted to the desired depth, a mechanical linkage, for example as shown at 64 on the actuator 60, is operated to cause a pop or frangible section 66, which is preferably a section of the shaft 34 of reduced strength, for example, formed by a precut slot or stress line in the shaft, to separate the proximal portion of the shaft 34 from the distal portion which is attached to the shaft 26.

The mechanical linkage 64 may be any type of suitable linkage. An embodiment is shown which is similar to that used to set the Li Medical “UltraFix” surgical anchor. The linkage shown causes the shaft 34 to be pulled in the direction of the arrow F. A suitable gripping mechanism, for example, a collet mechanism, grasps the shaft 34 at the portion 46. As shown in FIG. 3, the surgeon manipulates the mechanical linkage by outwardly moving portions 70 thereby to cause the actuator shaft to exert a force F in the proximal direction.

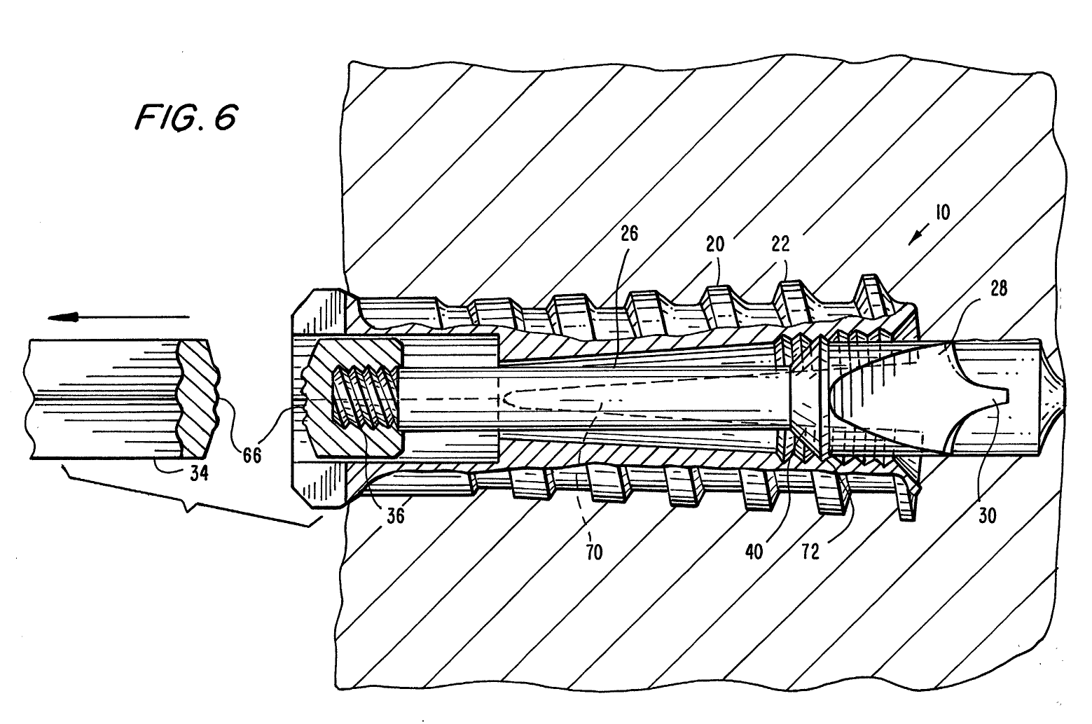

When the mechanical linkage 64 is actuated, it pulls the shaft 26 proximally, in the direction shown by arrow F, in the direction out of the bore hole, which causes the conical section 40 at the distal end of pin member 24 to move slidably against the tapered portion 42 of the outer members 20. This forces the outer member 20 radially outwardly by a camming action. The member 20 is slotted at its distal end as shown by longitudinal slots 70. Preferably at least two slots are employed. Additional slots can be employed. The slot or slots allow the member 20 to expand. Alternatively, member 20 may be unslotted but made of an expandable material. The distal to proximal motion in the direction of arrow F of shaft 26 causes the member 20 to expand radially outwardly. The conical portion 42 of the shaft 26 is provided with a ridge 72 which is captured by a one-way ratchet preferably comprising a plurality of annular grooves or serrations 44 in the internal diameter of outer member 20. The grooves 44 maintain the expansion of the screw member 20 in dependence on how far proximally the shaft 26 is moved before the pop section 66 snaps. The degree of expansion obtained is increased as the ridge 72 moves proximally, i.e., the more grooves that ridge 72 moves proximally past, the greater the degree of expansion of member 20. When a predetermined axial tensile force is applied by the actuating tool 60 to the shaft 34, the frangible portion 66 will snap, thus leaving the internal shaft 26 secured to the expanded external member 20 via a locking of the ridge 72 in one of the grooves of the one-way ratchet 44. The expansion of the outer member 20 securely locks the threads 22 of the fastener into the bone hole. As discussed, the member 20 does not require threads 22. Even without threads 22, the member 20 will be pressed into engagement with the bore hole walls thereby to secure it in position. FIG. 6 shows the fastening device in a deployed condition fixed in bone tissue. The fastening device 10 can have sutures secured thereto or can be used for the securement of a prosthesis.

The device according to the present invention can be modified in various ways. The device can be designed to have screw threads near the proximal end that are especially tailored for cortical bone purchase and the distal end may or may not have threads tailored for optimal cancellous bone purchase. The fastener may or may not have a drill bit end. If it does not have a drill bit end, a bore hole may be drilled initially and the fastener inserted into the predrilled hole.

The fastener can also use a different type of expansion device, for example a wedge or some other type of expansion device that is expanded by using axial force. Although the actuating device 60 has been described as employing a power drill, the actuating device can be powered manually. Further, a mechanical linkage has been shown which is employed to provide the rotational force for drilling and threading and the axial force F to cause the breakage of the frangible connection 66. Any other suitable actuating device can be employed.

An advantage of the invention is that fixation strength is increased due to the expansion effect, which is especially beneficial in soft bone. Another advantage is that the length of the bone fastener can be minimized since the design provides optimal cortical and cancellous bone fixation strength. Benefits of a shorter fastener are that it can be used in more applications and it affords a greater safety margin in terms of where it is placed.

As discussed, the present invention provides advantages over screw-in-screw type technology as shown, for example, in the Biedermann et al. patent. Because there are no internal threads, the fastener of the present invention utilizes less of the fastener cross section to apply the necessary axial force to expand the outer member 20. This leaves more material in the outer member 20 to resist clinical tension and bending stresses. This is shown in FIG. 5. FIG. 5A shows how the outer member 20 has a greater cross-sectional area than the screw-in-screw technology show in FIG. 5B. Thus, for anchors of equal diameter, the device of the present invention is superior in terms of clinical strength and more resistant to bending forces which could break the fastener.

Furthermore, the present invention has only frictional losses due to axial motion between the shaft and the outer member 20. Devices utilizing the screw-in-screw type of technology have additional frictional losses due to rotational friction in both the internal screw threads and the actuator. The additional losses require additional torque.

Furthermore, the present invention provides ease of use not provided by the screw-in-screw technology. After inserting the fastener, the invention requires only one quick motion to set the fastener. The screw-in-screw technology requires rotations which are fundamentally more time consuming. Additionally, the present invention requires no guessing when the job is done. The screw-in-screw technology results in the issue of whether the fastener has been tightened sufficiently. According to the present invention, once the frangible connection has snapped, the optimal setting of the anchor has been achieved.

Although the present invention has been described in relation to particular embodiments thereof, many other variations and modifications and other uses will become apparent to those skilled in the art. Therefore the present invention should be limited not by the specific disclosure herein, but only by the appended claims.

What is claimed is:

1. A surgical fixation device for fixation in a biological medium, the device comprising:
a first member having an internal longitudinal bore having an internal diameter;
a second shaft member slidably disposed in the internal bore of the first member, the second shaft member having a distal end having an external diameter greater than the internal diameter of the first member;
said distal end comprising a cam surface for engaging with an engaging surface of said first member;
said first member being expandable adjacent a distal end thereof;
said second shaft member having an engagement portion at a proximal end for engagement by an actuation tool for application of a proximally directed axial force to said shaft member;
whereby application of said proximally directed axial force to said shaft member causes said cam surface to slidably engage said engaging surface and thereby cause said first member to expand radially thereby securing said first member with said shaft member therein in the biological medium.

2. The device of claim 1, wherein said first member has external screw threads for screwing into the biological medium.

3. The device of claim 1, wherein said first member has at least one longitudinally extending slit adjacent the distal end to assist in expansion of the first member.

4. The device of claim 3, wherein the first member has at least two longitudinally extending slits adjacent the distal end.

5. The device of claim 1, wherein one of said second shaft member and said first member includes a drill bit at the distal end thereof for self drilling in the biological medium.

6. The device of claim 1, wherein said shaft has a frangible portion adapted to rupture upon application of a predetermined axial force to said shaft member.

7. The device of claim 1, wherein said shaft comprises a first distal portion and a second proximal portion, the first and second portions being mechanically coupled together.

8. The device of claim 2, wherein the shaft member is keyed to the first member, to allow rotation of the first member to drive the shaft member in rotation.

9. The device of claim 8, wherein the shaft member drives a drill bit disposed at the distal end thereof for self drilling into the biological medium.

10. The device of claim 1, further wherein the cam surface comprises a conical surface and the engaging surface comprises a tapering surface.

11. The device of claim 1, further comprising a locking member for locking the first member and second member in a defined relationship when the first member has expanded radially.

12. The device of claim 11, wherein the locking member comprises a ratchet.

13. The device of claim 12, wherein the ratchet comprises at least one groove provided in the internal diameter of the first member for securely engaging with a ridge provided on the shaft member.

14. The device of claim 13, wherein the at least one groove comprises an annular groove.

15. The device of claim 6, wherein the frangible portion comprises a portion of the shaft of reduced thickness.

16. The device of claim 7, wherein the first and second shaft portions are mechanically coupled by at least one of a screw connection and a crimped connection.

17. The device of claim 1, further comprising an actuating tool for applying said proximally directed axial force.

18. The device of claim 13, wherein the ratchet comprises a plurality of grooves along the internal diameter.

19. The device of claim 1, wherein the first member has an increasing outer diameter from the distal to proximal end thereof.

20. The device of claim 19, wherein the first member has an increasing outer diameter from the distal to proximal end thereof formed by the depth of the threads in the first member.

21. The device of claim 17, wherein the actuating tool includes a keyed distal end for driving the first member in rotation.

22. The device of claim 21, wherein the keyed distal end of the actuating tool comprises a screw driver projection and the proximal end of the first member includes a screw driver slot for engaging with the screw driver projection.

23. A method for delivering an expandable fixation device into biological tissue comprising the steps of:
accessing and preparing an insertion site in the biological tissue;
introducing an expandable fixation device into the biological tissue in an undeployed state;
the expandable fixation device comprising an outer sleeve and an inner shaft disposed longitudinally in the outer sleeve and having a distal end which has a larger diameter than an inside diameter of the distal end of the outer sleeve; and
once the expandable fixation device is disposed in the biological tissue at a selected depth, applying a proximally directed axial force to the inner shaft without rotation of the inner shaft to expand the distal end of the outer sleeve radially into the biological tissue thereby securing the expandable fixation device in the tissue.

24. The method of claim 23, wherein the outer sleeve has an external thread and the step of introducing the expandable fixation device comprises rotating said fixation device to cause it to screw into the biological tissue.

25. The method of claim 23, wherein the step of introducing the expandable fixation device comprises providing one of the inner shaft and the outer sleeve with a drill bit and rotating the fixation device to cause self drilling into the biological tissue.

26. The method of claim 23, further comprising applying the axial force to slidably move the distal end of the inner shaft against a surface of the outer sleeve to radially expand the outer sleeve.

27. The method of claim 26, further comprising providing a one-way ratchet to maintain expansion of said outer sleeve.

28. The method of claim 27, further comprising providing said axial force to the inner shaft to cause a frangible portion of said inner shaft to rupture leaving said outer sleeve and a portion of said inner shaft secured in position in the biological tissue.
